(19) United States
(12) Patent Application Publication
(10) Pub. No.: US 2025/0261881 A1
(43) Pub. Date: Aug. 21, 2025

(54) FLEXIBLE ELECTRONICS FOR ANALYTE DETECTION

(71) Applicants: Massachusetts Institute of Technology, Cambridge, MA (US); The Brigham and Women's Hospital, Inc., Boston, MA (US)

(72) Inventors: Carlo Giovanni Traverso, Newton, MA (US); Robert S. Langer, Newton, MA (US); Hen Wei Huang, Watertown, MA (US); Siheng You, Somerville, MA (US); Luca Di Tizio, Cambridge, MA (US)

(73) Assignees: Massachusetts Institute of Technology, Cambridge, MA (US); The Brigham and Women's Hospital, Inc., Boston, MA (US)

(21) Appl. No.: 18/859,331

(22) PCT Filed: Apr. 25, 2023
(86) PCT No.: PCT/US2023/019747

§ 371 Date: Oct. 23, 2024

Related U.S. Application Data
(60) Provisional application No. 63/337,822, filed on May 3, 2022.

(51) Int. Cl. A61B 5/145; A61B 5/00; A61B 5/1486

(52) U.S. Cl. CPC A61B 5/14532; A61B 5/1486; A61B 5/4839; A61B 5/686

(57) ABSTRACT

A glucose sensor is configured to be wrapped around a surface of an injection needle or cannula. The glucose sensor measures a glucose concentration of a patient when the injection needle or cannula is inserted into the patient. The glucose sensor includes a flexible substrate, at least two electrodes disposed on a surface of the flexible substrate, a glucose-responsive hydrogel at least partially disposed on a first electrode of the at least two electrodes, and a membrane permeable to glucose. The membrane is disposed on the glucose-responsive hydrogel. The total thickness of the glucose sensor is less than 10 pm.

### RELATED APPLICATIONS

This application claims priority under 35 U.S.C § 119(e) to U.S. Provisional Patent Application No. 63/337,822, filed May 3, 2022, and entitled “Flexible Electronics for Analyte Detection,” which is incorporated herein by reference in its entirety for all purposes.

### BACKGROUND

Diabetes mellitus is a chronic disease that affects 34 million people in the US and 422million people worldwide with rapidly increasing incidence rates. It is associated with significant morbidity and is one of the top ten leading causes of death worldwide. Glycemic control is a primary goal of therapy in diabetes, as it reduces complications, co-morbidities, and mortality, and insulin is one of the therapeutic classes used to achieve this. Insulin is a life-saving therapy in type 1 diabetes and is prescribed as stand-alone or combination therapy for type 2 diabetes, with approximately 25 percent of people with diabetes using insulin. Insulin use for glycemic control involves not only self-injections, often multiple per day, but also routine monitoring of glucose levels, commonly from finger stick capillary blood self-sampling. Moreover, insulin regimens can be extremely complex. A long-acting basal insulin is generally prescribed as a fixed dose taken once or twice daily. A different, short-acting, insulin can be used as a bolus both to mitigate the blood glucose rise after carbohydrate intake and as a correction for glucose levels above target.

The amount of insulin administered to dampen the glycemic excursion from an intake of carbohydrates is calculated using one or more factors. For example, the amount of insulin to be administered may be a fixed dose, may be based on the patient's current blood glucose concentration, and/or based on an insulin-to-carbohydrate ratio prescription. Calculating the dose using an insulin-to-carbohydrate ratio prescription offers more precision, since carbohydrate content per meal typically varies. The insulin amount used to correct an elevated glucose concentration varies with the amount of glucose elevation and the patient's insulin resistance. The insulin amount is also based on an estimation of how much one unit of insulin lowers blood glucose, sometimes called an insulin sensitivity factor, which is specific to an individual and can even vary with the time of day. These many factors place a significant burden on the patient (also referred to herein as a user) for blood glucose measurement and insulin delivery and can have a substantial effect on adherence.

The many devices that a patient uses and the several steps that the patient completes to safely administer a single insulin injection also places a significant burden on the patient. Typically, the patient typically performs multiple steps using three or more different devices to administer a safe and appropriate amount of short-acting insulin. Specifically, a patient typically uses a lancing needle to puncture their skin and extract a droplet of blood. The patient prepares a glucometer with a single-use testing strip and places their blood droplet on the testing strip, so that the glucometer can measure their blood glucose concentration. Once the patient knows their blood glucose concentration, they calculate how much insulin to administer, corrected for several factors including their present glucose level and their insulin resistance. The patient prepares an insulin pen or syringe with a single-use needle and then performs the self-injection with the needle and administers the insulin with the syringe or insulin pen. A patient typically performs this painful multi-step procedure three or more times daily.

Several technologies were developed recently to help patients manage diabetes. Insulin pumps, for example, automate insulin dose calculations and can deliver insulin both continuously and on demand. Continuous glucose monitoring (CGM) technology has reduced the number of finger sticks and revolutionized the amount of blood glucose data that can be collected. More recently, hybrid devices, also known as artificial pancreases, have been approved that both monitor glucose and deliver insulin automatically. However, these devices are expensive, require extensive patient training, have to be worn continuously, and are accessible to a small percentage of patients, generally those with type 1 diabetes. Many insurance providers do not cover CGM technology for patients. A patient's lifestyle choices may preclude using a CGM, and a CGM may not be worn during a limited number of activities.

### SUMMARY

Embodiments of the present technology include a glucose sensor configured to be wrapped around a surface of an injection needle or cannula. The glucose sensor includes a flexible substrate, at least two electrodes disposed on a surface of the flexible substrate, a glucose-responsive hydrogel at least partially disposed on a first electrode of the at least two electrodes, and a membrane permeable to glucose. The membrane is disposed on the glucose-responsive hydrogel. The total thickness of the glucose sensor is less than 100 μm.

In a version, the glucose sensor is configured to measure a glucose concentration up to about 26 mM. The glucose sensor may be capable of measuring a glucose concentration of a patient when the injection needle or cannula is inserted into the patient in less than 40 seconds after the injection needle or cannula is inserted.

The glucose-responsive hydrogel may include a polymer permeable to glucose, an oxidoreductase enzyme that catalyzes oxidation of glucose, and a redox mediator. The polymer may include at least one of chitosan, polyvinyl alcohol, or Nafion. The oxidoreductase enzyme may include at least one of glucose oxidase or a derivative or mutant thereof. The redox mediator may include at least one of an iron salt complex, an osmium salt complex, or a ruthenium salt complex. The iron salt complex, if present, may include potassium ferricyanide. The ruthenium salt complex, if present, may include hexamine ruthenium.

The at least two electrodes may include a noble metal. The membrane may include Nafion. A second electrode in the at least two electrodes may be a reference electrode that includes silver. The flexible substrate may include at least one of polydimethylsiloxane or polyimide. The flexible substrate may be a first flexible substrate and the sensor may include a second flexible substrate disposed on at least a portion of the first flexible substrate, the second flexible substrate having voids where the electrodes are located.

The glucose sensor may include the injection, where the flexible substrate is disposed on a shaft of the injection needle. The glucose sensor, including the injection needle, may be part of an “all-in-one” insulin pen.

Another embodiment of the present technology includes a method of measuring a glucose concentration of a patient. The method includes inserting an injection needle into the patient, where the injection needle is fluidically coupled to a container holding a medicine. The method also includes measuring a glucose concentration of the patient from an interstitial fluid of the patient in less than 40 seconds after the injection needle is inserted into the patient using a glucose sensor wrapped around an outer surface of the injection needle.

In a version, the method further includes administering the medicine to the patient through the injection needle if the glucose concentration measured by the glucose sensor during the step of measuring is outside of a euglycemic range. The step of administering is performed without withdrawing the needle after the step of measuring.

The step of measuring may include operating the glucose sensor at a voltage of about −0.4 V to about 0.4 V. The step of measuring may include measuring a glucose concentration of up to about 26 mM.

Another embodiment of the present technology is a glucose sensor wrapped around an outer surface of an injection needle. The glucose sensor includes a flexible polymer substrate, a membrane permeable to glucose, and a patterned metal electrode disposed on the flexible polymer substrate and coated with a glucose-responsive hydrogel. The membrane is disposed on the flexible polymer substrate. The glucose-responsive hydrogel includes glucose oxidase, chitosan, and hexamine ruthenium. The glucose sensor is capable of measuring a one-time glucose concentration of a patient when the injection needle is inserted into the patient. The glucose sensor is capable of measuring the one-time glucose concentration in less than 40 seconds after the injection needle is inserted into a patient.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1 shows a cross-sectional view of a section of a flexible glucose sensor.

FIG. 2 shows an array of fabricated flexible electrodes for flexible glucose sensors.

FIG. 3 shows an exploded view of the fabrication layers of an array of flexible glucose sensors.

FIG. 4 shows a flexible glucose sensor wrapped around the shaft of an 18-gauge injection needle forming an all-in-one needle.

FIG. 5 shows a flexible glucose sensor wrapped around the shaft of a 26-gauge injection needle forming an all-in-one needle.

FIG. 6 shows an all-in-one needle inserted into subcutaneous tissue.

FIG. 7 shows a fabrication scheme for making an array of flexible electrodes for flexible glucose sensors.

FIG. 8 shows the lift-off process for removing a fabrication substrate from the array of flexible electrodes.

FIG. 9A shows an exploded view of an all-in-one insulin pen with the all-in-one needle.

FIG. 9B shows the needle hub of the all-in-one needle.

FIG. 9C shows the all-in-one needle mounted in the needle hub shown in FIG. 9B.

FIG. 10 shows a side view of the all-in-one insulin pen with the all-in-one needle.

FIG. 11 shows the all-in-one insulin pen held in a hand.

FIG. 12 shows cyclic voltammetry results of a glucose sensor in a 12 mM glucose solution and in a phosphate buffered saline (PBS).

FIG. 13 shows chronoamperometric current measurements versus glucose concentration using a glucose sensor.

FIG. 14A shows chronoamperometric current measurements versus glucose concentration using the flexible glucose sensor 5 seconds after sensor wetting.

FIG. 14B shows chronoamperometric current measurements versus glucose concentration using the flexible glucose sensor 10 seconds after sensor wetting.

FIG. 15A shows transient currents measured with an all-in-one needle operating at different voltages.

FIG. 15B shows stabilization time of an all-in-one needle measuring different glucose concentrations.

FIG. 16A shows cross sensitivity tests of the flexible glucose sensor.

FIG. 16B shows interferents used in the cross sensitivity tests in FIG. 16A.

FIG. 17A is a schematic representation of the experimental setup for in vivo testing of the all-in-one needle in a porcine model.

FIG. 17B shows a current magnitude vs. time plot showing a glucose measurement with an all-in-one needle in vivo.

FIG. 17C shows mean current magnitude 5-10 seconds post insertion versus measured blood glucose concentration for N=14 separate animal measurements.

FIG. 18 shows recordings of current versus time recorded using the all-in-one needle prior to (blood glucose (BG)=42 mg/dL) and following dextrose injection (BG=105 mg/dL in a non-diabetic animal.

FIG. 19 shows ruthenium (Ru) dissolution from the flexible glucose sensor.

### DETAILED DESCRIPTION

The flexible glucose sensor (also called the sensor or glucose sensor) described herein provides an avenue for shifting away from the current standard of care for managing diabetes using several devices including a lancet, a glucose monitor with a testing strip, and an insulin syringe or pen with an injection needle. Because of the glucose sensor's flexibility, it can be combined with the injection needle or cannula used to administer the insulin. For example, the flexible glucose sensor can be disposed on a curved outer surface of the injection needle's shaft to create an “all-in-one” needle that can both measure glucose levels and administer a medicine (e.g., insulin or glucagon) when the needle is inserted into the patient. Since the glucose sensor is on the needle's shaft, it does not affect the needle's ability to administer the medicine from its tip.

Because the flexible glucose sensor uses relatively cheap components, in one example it can be disposed on a disposable, one-time use injection needle, and discarded along with the needle after its one-time use to measure glucose concentration. As another example, the flexible glucose sensor can be disposed on a cannula used to deliver a medicine (e.g., as part of a continuous glucose monitor (CGM) or insulin pump system). The flexible glucose sensor has a total thickness less than about 500 μm (e.g., 500 μm, 400 μm, 300 μm, 200 μm, or 100 μm) and adheres strongly to the needle or cannula to avoid or substantially reduce delamination of the sensor from the needle or cannula surface during or after the needle or cannula is inserted into the tissue.

The flexible glucose sensor measures glucose concentrations in any biological or otherwise aqueous fluid. When the flexible glucose sensor is part of the “all-in-one” needle, it can measure glucose concentrations in the biological fluid that it is exposed to when the needle is inserted into a patient's tissue. As an example, the biological fluid may be interstitial fluid. The glucose content of interstitial fluid is proportional to that of blood glucose content and can be used to determine the blood glucose concentration. As another example, the biological fluid may be blood and the glucose sensor may measure a blood glucose concentration directly.

In one version, the flexible glucose sensor may measure a single, one-time measurement of the patient's glucose concentration. For example, a single measurement may be preferable when the glucose sensor is used as part of an “all-in-one” needle. In another version, the flexible glucose sensor may measure a series of measurements of the patient's glucose concentration, so long as the needle or cannula upon which the glucose sensor is disposed is still inserted in the patient's tissue. For example, a series of measurements may be preferable when the glucose sensor is disposed on a cannula residing in the patient's tissue over a long period of time.

Having the glucose sensor disposed on a surface of the injection needle or cannula, provides several benefits to the patient. One of these benefits is having fewer devices to carry around to manage one's diabetes. Instead of carrying three separate devices, the patient can carry a single “all-in-one” device that provides glucose measurements and medicine administration, thereby increasing convenience. Another benefit is using fewer steps to test one's blood glucose concentration and administer insulin. Conventionally, this sequence of steps includes separate steps for skin preparation, lancing, blood draw, blood glucose measurement, insulin dose calculation based on blood glucose measurement, and insulin administration. The all-in-one device with a flexible glucose sensor on the injection needle or cannula produces the same outcome as the conventional sequence but without the steps of skin preparation, lancing, and blood draw. Reducing the number of steps increases convenience to the patient and reduces total time for diabetes management. Another benefit is decreasing the number of skin punctures during each insulin administration sequence from two to one since the all-in-one device forgoes the step of lancing the skin to collect blood for a glucose measurement. In doing so, the all-in-one device decreases the patient's pain and discomfort in managing their diabetes. All together, these benefits can increase adherence in diabetes management.

In one embodiment, the flexible glucose sensor includes a working electrode in operative communication with a glucose-responsive hydrogel. The glucose-responsive hydrogel may be disposed on at least a portion of the working electrode. The glucose-responsive hydrogel is permeable to glucose and comprises a molecule or enzyme that catalyzes the oxidation of glucose (e.g., an oxidoreductase enzyme) to produce a current that is directly related to the concentration of glucose in the environment of the glucose-responsive hydrogel. The working electrode includes a biocompatible metal that has a thickness small enough so that the working electrode is sufficiently flexible to allow the glucose sensor to be conformally disposed on an arbitrary three-dimensional shape having bends with bend radii as small as about 0.08 mm. A semi-permeable membrane may be disposed on the glucose-responsive hydrogel that is permeable to glucose.

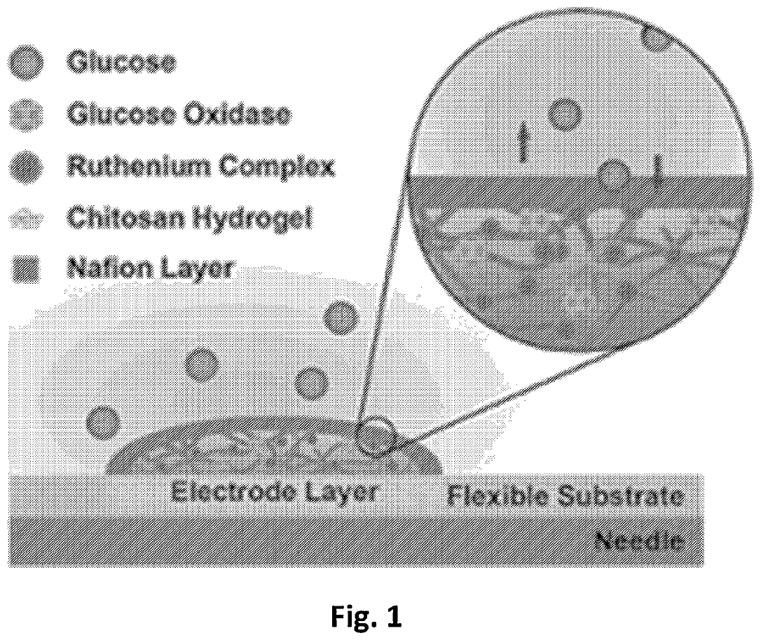

FIG. 1 shows a cross-section view of part of a flexible glucose sensor disposed on a surface of a needle. The working electrode is disposed on a substrate (e.g., a polymer) that is thin, flexible, and electrically-insulating. The working electrode is one or more biocompatible, conductive metals (e.g., gold, silver, platinum, nickel, or palladium) or carbon. A glucose-responsive hydrogel is disposed on a surface of the working electrode having a thickness of about 10 μm to about 100 μm (e.g., 10 μm, 20 μm, 30 μm, 40 μm, 50 μm, 60 μm, 70 μm, 80 μm, 90 μm, or 100 μm). The hydrogel immobilizes a redox mediator and glucose oxidase near the surface of the working electrode for glucose sensing.

The hydrogel includes a polymeric material, a redox mediator, and an enzyme that selectively catalyzes the oxidation of glucose (e.g., an oxidoreductase enzyme). Optionally, the hydrogel may include a humectant (e.g., sugar alcohol, propylene glycol, or lactic acid). The polymeric material depicted is chitosan, but other polymeric materials may be used instead of or in addition to chitosan, including polyvinyl alcohol (PVA), fluoropolymers (e.g., Nafion), or a combination thereof. For example, the polymeric material may include chitosan in a weight percent of about 10% to about 80%, PVA in a weight percent of about 10% to about 80%, and fluoropolymer in a weight percent of about 1% to about 5%.

The redox mediator (also called an electrochemical mediator) depicted is hexamine ruthenium, but other mediators that act as electron shuttles between glucose oxidase and the electrode may be used instead of or in addition to hexamine ruthenium, including iron salt complexes, osmium salt complexes, and/or other ruthenium salt complexes. The amount of redox mediator in the hydrogel is about 2 μg to about 10 μg in a 6 μL volume of hydrogel. As an example, the iron salt complex may include potassium ferricyanide. The enzyme depicted is glucose oxidase (GOₓ) but other enzymes may be used instead of or in addition to GOₓ, including any derivatives or mutants of GOₓ, or glucose dehydrogenase. For example, a mutant of glucose oxidase may have a higher activity than glucose oxidase, making it favorable for sensing.

A semi-permeable membrane is disposed on the outer surface of the hydrogel. The semi-permeable membrane prevents or substantially reduces interaction between interferents in the biological fluid (e.g., anions) and the sensor, while also being permeable to glucose. The semi-permeable membrane is permeable to glucose and non-permeable to interferents. The semi-permeable membrane may cover the hydrogel so that no surface of the hydrogel is directly exposed to biological fluid during operation. The semi-permeable depicted is Nafion but other polymers may be used instead of or in addition to Nafion, including polyurethanes and other fluorinated hydrocarbons.

In one example, the glucose-responsive hydrogel was prepared by mixing a 30 mg/mL±20 mg/mL hexamine ruthenium in chitosan (0.1% to 0.5% by weight chitosan in 1% to 5% by weight acetic acid) solution by stirring the solution at 300 rpm and heating the solution at 80° C. for 4 hours. 4 μL of NaOH (2 M), 5 μL of glucose oxidase (GOₓ) in water (10 mg/mL to 100 mg/mL, e.g., 50 mg/mL) and 10 μL of glutaraldehyde (0.1% to 5% by weight, e.g., 1% by weight, in water) were added to 100 μL of the hexamine ruthenium and chitosan solution to form the final solution. Three layers of 2 μL each of the final solution were drop casted on the working electrode while waiting 30 minutes for each layer to dry to form the glucose-responsive hydrogel. Subsequently a 1.5 μL layer of Nafion (0.5% in water) was drop cast and left to dry for 30 minutes to form the semi-permeable membrane. Besides drop casting, the hydrogel may also be prepared on the working electrode using spray coating, dip coating, liquid printing, or silk screen printing.

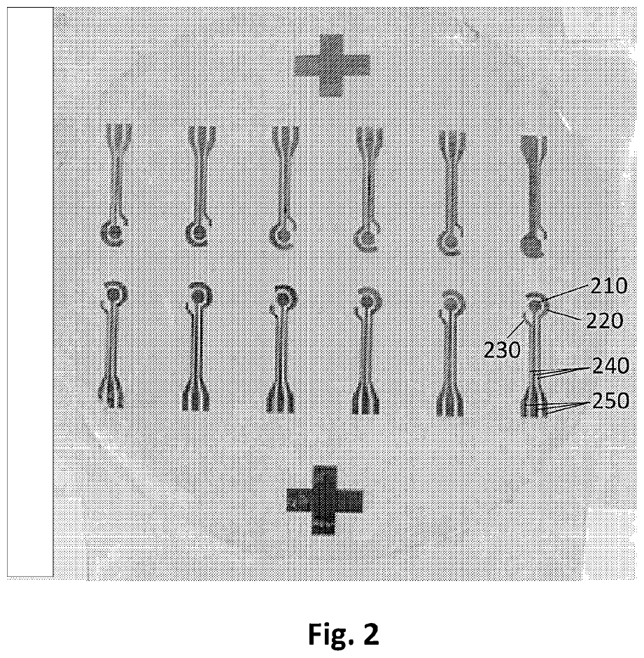

The flexible glucose sensor may include additional structures to increase fluid contact with the surface of the flexible glucose sensor. For example, surface structuring may be used to increase fluid contact. Surface structuring may include the hydrogel, the electrodes, and/or the needle surface having an increased surface roughness of about 1 μm to about 1,000 μm. As another example, the electrodes may have a three dimensional shape to increase fluid contact. As another example, microfluidic channels may be used to guide fluid to the glucose-responsive hydrogel. The microfluidic channels may have a diameter of about 10 μm to about 1,000 μm. FIG. 2 shows an array of fabricated flexible electrodes for flexible glucose sensors. The scale bar is 10 mm. Twelve sets of electrodes are shown. As described below, the electrodes may be fabricated using a batch microfabrication process to produce many sets of electrodes at one time. Each set may include a working electrode, counter electrode, and a reference electrode. In one version, each set includes a working electrode and a counter electrode, but no separate reference electrode, and the counter electrode also acts as the reference electrode. Three electrode sets may be preferred because they mimic the electrode configuration in commercial glucose test strips. The working electrode, counter electrode, and reference are biocompatible, conductive metal (e.g., gold, silver, platinum, or palladium) or carbon. The glucose-responsive hydrogel is disposed on top of the working electrode before use. The reference electrode, if present as a separate electrode, may be metal or coated with a silver/silver chloride paste. An all-in-one needle may include one or several sets of electrodes. The electrodes each have a thickness of about 50 nm to about 500 nm. The electrodes may have a circular, concentric, or interdigitated shape. If interdigitated, the electrode fingers have widths of about 10 μm to about 1 mm.

In FIG. 2, the working electrode 210 is circular, the counter electrode 220 is arc shaped and concentric with the working electrode 210. The reference electrode 230 is curved. Conductive leads 240 connect the electrodes 210, 220, and 230 to conductive pads 250 that are used to electrically couple the electrode sets to electrical measurement hardware.

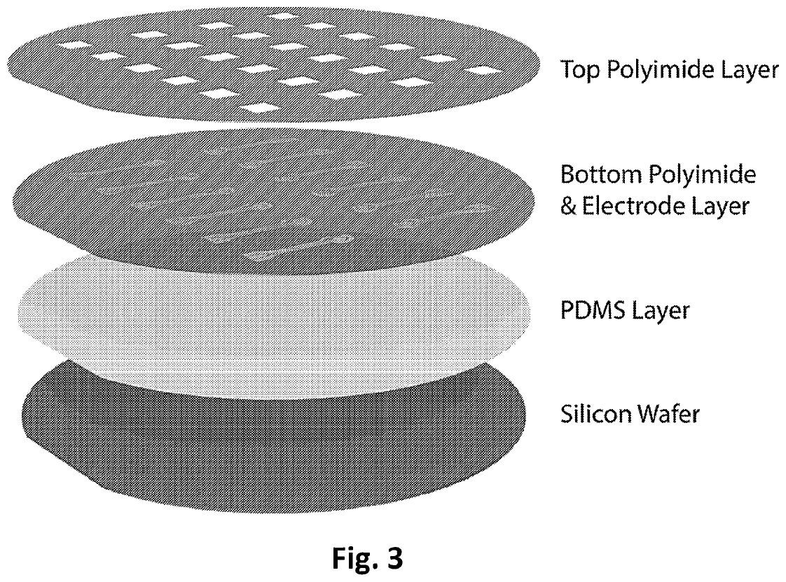

FIG. 3 shows an exploded view of the layers of an array of flexible glucose sensors. The layers of the flexible glucose sensors may be fabricated on a silicon wafer substrate and released from the silicon wafer before use. The glucose sensor may include a base layer selected to adhere to the surface of a needle, for example a silicon-based organic polymer (e.g., polydimethylsiloxane, PDMS), silicon dioxide, or another inorganic oxide or organic silicone polymer that can undergo silicone bonding. The base layer improves ease of fabrication by providing adhesion and/or bonding between the flexible glucose sensor layers and the silicon wafer. On top of the base layer is a bottom polymer layer that is electrically insulating and provides electrical passivation. The bottom and top polymer layers may be polyimide, polyurethane, polyethylene terephthalate, polyethylene naphtholate, or SU-8 photoresist. As an example, the base layer may be polyimide. The metallic layer includes electrodes and interconnects and is disposed on the bottom polymer layer in the configuration described with respect to FIG. 2. As an example, the metallic layer may include gold, platinum, or another noble metal. A top polymer layer is disposed on the at least part of the bottom polymer layer and part of the metallic layer. The top polymer layer includes voids (also called windows) so that the top polymer layer is not disposed on a large portion, or all of the electrodes surfaces and interconnect surfaces. The voids may have the same areal dimensions as the electrodes or may have an area up to about 10% larger (e.g., about 5% to about 10% larger) than the electrodes. In this way, the top polymer layer leaves these regions of the metallic layer exposed so that the electrodes can be used for glucose measurements and the interconnects can electrically couple the electrodes to electrical measurement hardware (e.g., a potentiostat and/or a galvanostat). As an example, the top polymer layer may include polyimide having a thickness of about 0.5 μm to about 10 μm. The top polymer layer, bottom polymer layer, and metallic layer are all made of biocompatible materials.

The electrodes may be functionalized before or after the top polymer layer is placed. The working electrodes are functionalized with the glucose-responsive hydrogel described above with respect to FIG. 1. The reference electrodes are functionalized with silver/silver chloride, silver, or gold. For example, the reference electrode may be functionalized by depositing an Ag/AgCl paste onto the surface of a biocompatible metal electrode and then cured to form an Ag/AgCl reference electrode. In one example, 1 μL of Ag/AgCl paste was applied to the reference electrode using a pipette and annealed at 100° C. for 1 hour. The counter electrodes are not functionalized.

Following fabrication, if the arrangement of layers includes an array of electrode sets it can be cut into sections of one or more electrode sets that will be placed on the injection needle or cannula. The arrangement of layers may be removed or peeled off from the silicon wafer substrate. In one example, the layer was removed using a water-soluble transfer tape. The assembled layers were cut into shapes (e.g., rectangles with a dimension matching or smaller than the shaft of the target needle or cannula) that include one or more sets of electrodes to be placed on the target needle or cannula. The one or more sets of electrodes with their accompanying layers can then be transferred onto and adhered to the surface of a needle or cannula, where the transfer does not block the channel of the needle or cannula. Once transferred, the flexible glucose sensor is adhered to the shaft of the needle or cannula using silicone bonding, polycaprolactone adhesive, cyanoacrylate glue, polyurethane adhesive, or another biocompatible adhesive.

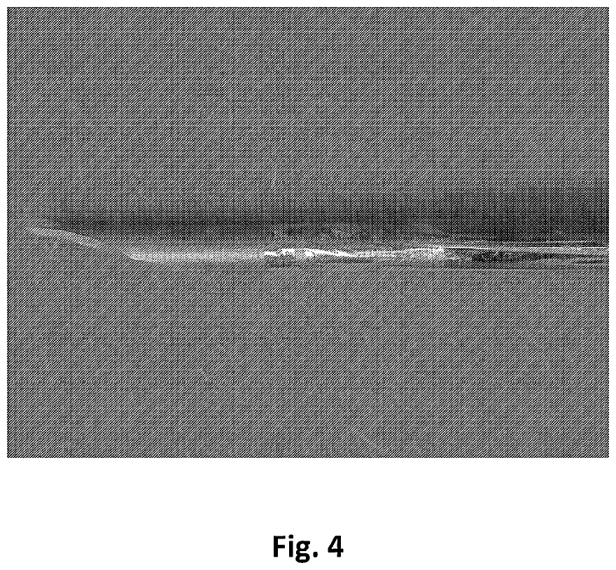

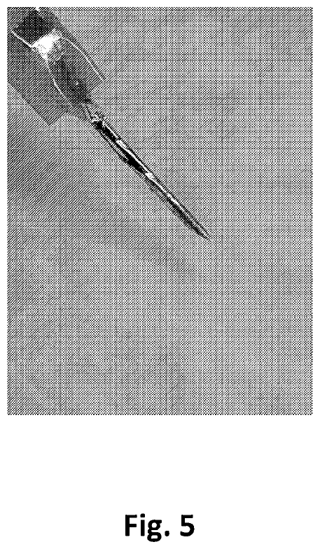

FIG. 4 shows a flexible glucose sensor wrapped around the shaft of an 18-gauge (18G) hypodermic injection needle forming an all-in-one needle. FIG. 5 shows a flexible glucose sensor wrapped around the shaft of a 26-gauge (26G) injection needle forming an all-in-one needle. A flexible glucose sensor may be disposed on an injection needle or cannula having a needle gauge of about 18G to about 30G (e.g., 18G, 19G, 20G, 21G, 22G, 23G, 24G, 25G, 26G, 27G, 28G, 29G, or 30G) and a length long enough to deliver insulin. Multiple sensors may be arranged on a needle along the length and/or radius of the needle. The one or more sensors may be placed near the tip of the needle to access the subcutaneous region when the needle is injected.

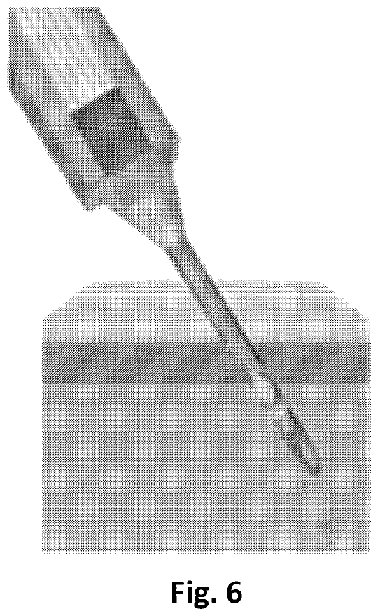

FIG. 6 shows an all-in-one needle inserted into subcutaneous tissue. When the all-in-one needle is inserted into the subcutaneous tissue, the flexible glucose sensor can measure the glucose concentration in the interstitial fluid in the subcutaneous space, and this measurement can be used to determine the patient's blood glucose concentration. Simultaneously while the sensor is measuring glucose, before the sensor measures glucose concentration, or after the sensor measures glucose concentration, the all-in-one needle can be used to deliver a medicine into the subcutancous tissue. Preferably, the sensor measures the glucose concentration prior to medicine administration, and the glucose concentration measured is used to determine the amount of medicine (also called a dose) to be administered to the patient.

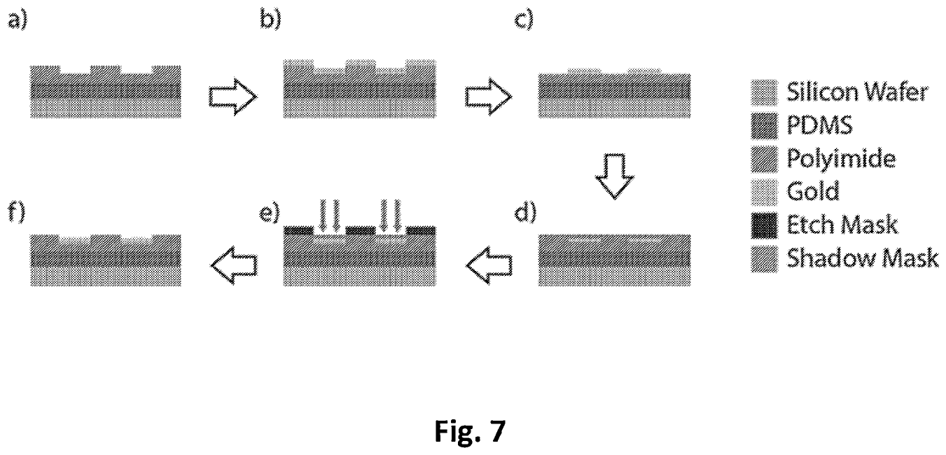

FIG. 7 shows an example fabrication scheme for making the arrangement of layers with an array of flexible electrodes for flexible glucose sensors. In this example, a 3-inch diameter silicon oxide wafer was silanized in a vacuum chamber under vacuum in the presence of a drop of 1H,1H,2H,2H-perfluorooctyltrichlorosilane for 20 minutes. Subsequently, the silane bonding was finalized by baking the wafer at 120° C. for at least 3 minutes. The silanized wafer was thoroughly cleaned using acetone and isopropyl alcohol. PDMS (SYLGARD 184 silicone) was prepared by mixing base with curing agent at a ratio of 10:1 and, the mixture was spin-coated onto the silanized wafer at 500 rpm for 5 seconds followed by 4000 rpm for 45 seconds. The PDMS-coated wafer was baked in an oven at 65° C. for at least 12 hours.

The bottom polymer layer in this example was polyimide. Polyimide solution was prepared by mixing VTEC PI-1388 with 1-methyl-2-pyrrolidinone at a ratio of 2:1 at room temperature using a Speedmixer. The PDMS-coated wafer was exposed to an O₂ plasma (100 W, 50 sccm, 30 s) surface treatment. The prepared polyimide solution was then spin coated onto the wafer at 4000 rpm for 45 seconds. The wafer was then soft-baked at 65° C. for 4 minutes before it was gradually heated to 200° C. for a 2-hour hard bake. The wafer was then allowed to cool to room temperature gradually to avoid film cracking due to temperature shock.

The metallic layer including the electrodes was deposited onto the bottom polyimide layer using a mask. For electrodes designed for 18G needles a laser cut patterned Kapton shadow mask was used. For electrodes designed for 25G needles, a photoresist mask of LOR3A was prepared by spin-coating it onto the bottom polyimide layer at 4000 rpm. The layers were then baked at 115° C. for 4 minutes. S1805 was then spin-coated onto the LOR3A layer at 4000 rpm, and again baked at 115° C. for 4 minutes. The S1805 surface was then exposed to the design pattern using a maskless aligner (e.g., Heidelberg MLA150). Using either the Kapton shadow mask or the photoresist mask, the wafer was then loaded into an electron beam evaporator where 5 nm of titanium or chromium was deposited onto mask surface, followed by 100 nm of Au. The titanium or chromium layer promoted adhesion of the Au layer to the substrate. The mask was then removed to reveal the patterned metallic layer including electrodes and interconnects. The photoresist mask was lifted off by immersing the wafers in acetone for about 4 hours. The Kapton shadow mask was simply lifted off of the surface of the wafer.

The top polymer layer was also a polyimide layer. The top polyimide layer was deposited onto the metallic and exposed bottom polyimide layers using the same spin-coating process as described above with respect to the bottom polyimide layer. To create the voids or windows in the top polyimide layer, the top polyimide layer was etched. An etch mask was fabricated using a first method or a second method. The first method included spin-coating S1822 onto the top polyimide layer on the wafer at 3000 rpm for 45 seconds and then baked at 115° C. for 3 minutes. Etch windows in the S1822 layer were created using photolithography. The second method included depositing nickel using a laser cut Kapton shadow mask. Once the mask was created, the exposed portions of the top polyimide layer were etched to expose metallic electrodes and interconnects (also called input/output pads) using reactive ion etching in a gas ratio of 5:2 O₂:CF₄. Following etching, the etch mask was removed to form the final assembly of layers of the flexible glucose sensor. The S1822 etch mask was removed using acetone and the nickel etch mask was removed using nickel etchant (e.g., TFB).

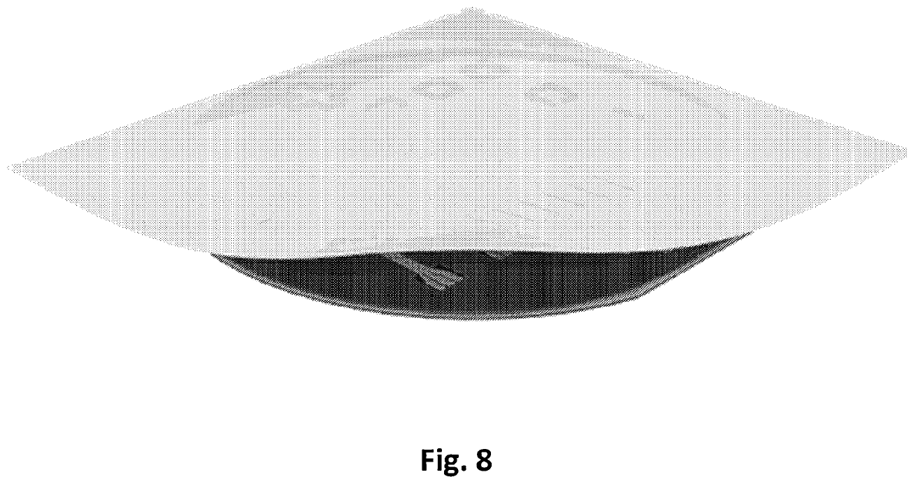

FIG. 8 shows an example lift-off process for removing a fabrication substrate (e.g., a silicon wafer) from the layer arrangement including the array of flexible electrodes. Water soluble transfer tape was applied to the surface of the top polyimide layer in the assembled layers on the silicon wafer. The silicon wafer was then cut using a diamond scribe on its back surface opposite the surface on which the assembled layers are disposed. Cutting the wafer made it easier to peel the electrodes/transfer tape from the wafer.

Once the layer arrangement was lifted off of fabrication substrate, it was applied to an injectable needle or cannula. As an example, a commercial luer-lock needle with a 3D printed needle hub was coated with silicone and exposed to a corona plasma treatment to facilitate electrode bonding. The electrode set layer arrangement was floated on the surface of water until the water-soluble transfer tape was dissolved, and the needle was aligned with the electrode pattern and transferred. Prior to applying the electrode set layer arrangement to the needle, the exposed PDMS surface in the assembled layers was treated with a plasma (e.g., corona plasma) to increase adhesion between the PDMS layer on the sensor and the PDMS layer on the needle or cannula shaft. The assembled all-in-one needle was baked in a vacuum oven at 80° C. to tightly bond the silicone layers on the needle to those on the layer arrangement.

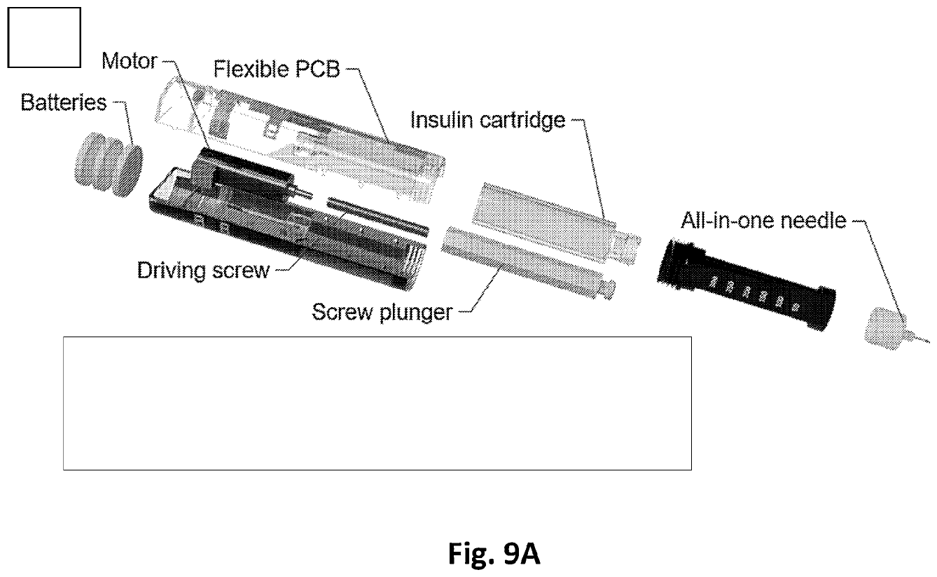

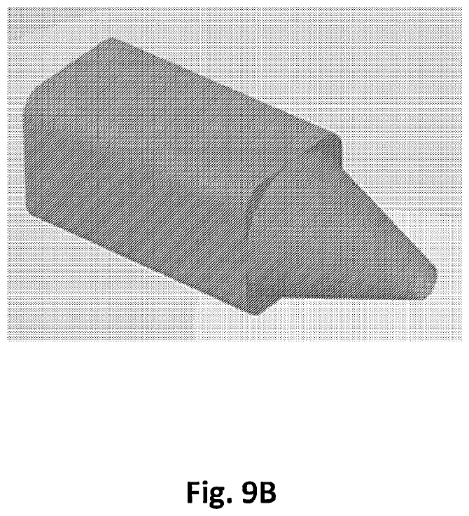

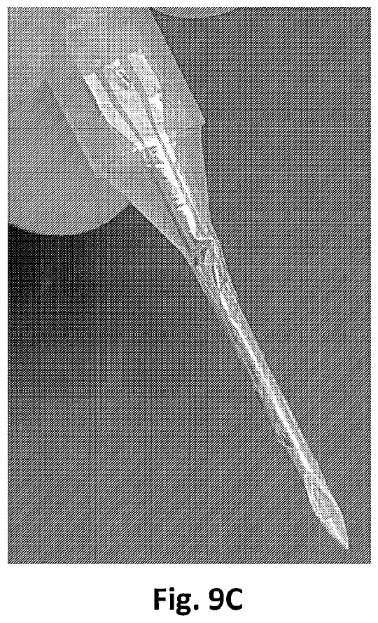

FIG. 9A shows an exploded view of an all-in-one insulin pen with the all-in-one needle. FIG. 9B shows a needle hub that may hold the all-in-one needle and may electrically couple the flexible glucose sensor to a potentiostat or a galvanostat in the all-in-one insulin pen. The conductive pads on the flexible glucose sensor were electrically coupled to the potentiostat or galvanostat via conductive tape (e.g., anisotropic conductive tape) and flexible cable (e.g., Premo-flex cable). FIG. 9C shows the all-in-one needle mounted into the needle hub. FIG. 9C shows part of the flexible glucose sensor's electrical leads all of its conductive pads enclosed in the needle hub where the conductive pads couple with the conductive tape.

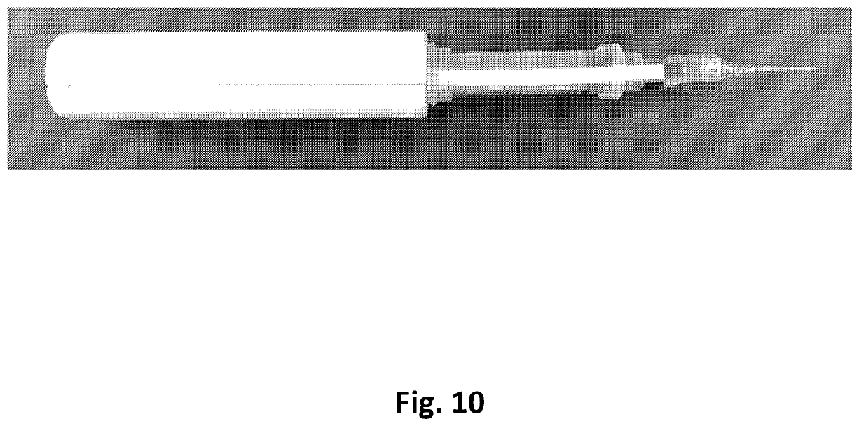

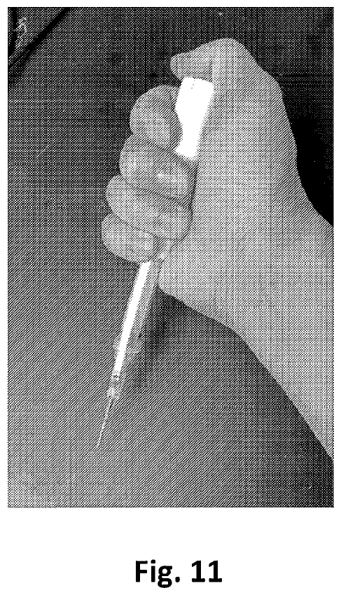

FIG. 10 shows a side view of the all-in-one insulin pen with the all-in-one needle shown in FIG. 9A. FIG. 11 shows the all-in-one insulin pen held in a hand. The all-in-one needle can be mechanically and fluidically coupled to an insulin pen or a syringe used to administer insulin or another medicine. An all-in-one insulin pen using the all-in-one needle includes a microcontroller, potentiostat (e.g., an Analog Front End potentiostat), insulin pump, rechargeable battery, and a wireless communication module (e.g., a Bluetooth module). Together, the all-in-one insulin pen includes the all-in-one needle, which has a flexible glucose sensor on its shaft. Anisotropic conducting film is used to electrically couple the conductive pads on the flexible glucose sensor to a flat flexible cable that electrically couples with the potentiostat. The all-in-one pen is capable of sensing the patient's interstitial glucose concentration, calculating an insulin dose to be delivered to the patient (e.g., by factoring in the current glucose concentration), and delivering the insulin dose calculated. These steps may be used to deliver a pre-prandial insulin dose to a patient. The all-in-one needle measures interstitial glucose concentrations in less than 40 seconds after the needle is inserted.

The all-in-one pen may include a display to display the glucose concentration measured by the all-in-one needle and potentiostat. By displaying the glucose concentration measured, the patient or a healthcare practitioner can see the measured concentration and use it to manually calculate an appropriate insulin dose accordingly. The display may be an OLED, LED, or LCD screen. Alternatively, or additionally, the microcontroller may use the measured glucose concentration to automatically calculate an appropriate insulin dose to be delivered to the patient. Alternatively, or additionally, the wireless communication module may wirelessly send the glucose measurement to an external device (e.g., a smart phone, a tablet, or another type of computer). The external device may then display the glucose measurement to the patient or a healthcare practitioner so that they can determine an appropriate insulin dose accordingly. Alternatively, or additionally, the external device may automatically calculate an appropriate insulin dose to be delivery to the patient and wirelessly transmit that information to the all-in-one pen via its wireless communication module. The external device may also record and store a log or history of glucose measurements. The external device may be in the possession of the patient or their healthcare practitioner.

The insulin dose calculation encoded in the all-in-one pen system is used to correct for high blood sugar based on the glucose measurement while the calculation encoded in the external device may consider additional factors from the patient or healthcare practitioner, including the patient's carbohydrate intake. The patient may manually input an insulin sensitivity factor and an insulin to carbohydrate ratio in the all-in-one pen system and/or the external device as an initialization setting.

The insulin dose is calculated directly by a microcontroller in the all-in-one pen, manually be the patient or healthcare practitioner, or using an external device (e.g., a smart phone) connected to the all-in-one pen via a wireless communication system in the pen. The all-in-one insulin pen then delivers the calculated insulin dose to the patient. The insulin pump includes a syringe-like insulin cartridge (also called a container), a stepper motor driver with a screw plunger, a stepper motor, and a stepper motor holder. As an example, the stepper motor may be a two-phase four-wire geared stepper motor with a lead screw. Electrical conductors (e.g., electrical wires or ribbon cables) electrically couple the stepper motor to the microcontroller so that the microcontroller can control the insulin dose.

With the needle still in the tissue, the stepper motor drives the stepper motor driver to administer insulin from the cartridge (also called a reservoir) to the patient via the insulin needle. The insulin cartridge, stepper motor driver, and stepper motor holder may be 3D printed using durable materials (e.g., nylon, thermoplastic polyurethane, polylactic acid, acrylonitrile butadiene styrene, polyethylene terephthalate glycol, polyvinyl alcohol, or high impact polystyrene). Pumping rate is controlled by changing the speed of the stepper motor. The insulin cartridge may be pre-filled with insulin (e.g., from a commercially available insulin vial using a syringe). The cartridge may be loaded into the all-in-one pen before use. The amount of insulin delivered may be displayed on the pen's display so that the patient can know how much insulin is being delivered. The amount of insulin delivered may be wirelessly transmitted to the external device via the wireless communication module so that this information may be stored as art of the insulin dose history.

All of the electronic components in the all-in-one pen may be powered by one or more rechargeable batteries. As an example, the rechargeable battery may be a lithium-ion polymer (LIPO) battery. As an example, the rechargeable battery may be recharged by the patient or a healthcare professional when desired by electrically coupling the all-in-one pen to an external power source directly or using inductive coupling.

The all-in-one pen may be assembled by operably coupling the microcontroller with the potentiostat, insulin pump stepper motor, and wireless communication system. All of these components may be assembled into a handheld housing.

The all-in-one pen using the flexible glucose sensor substantially decreases the time expenditure and simplifies the process for blood sugar measurement and insulin delivery. This conventionally time-consuming process typically uses several different devices. In contrast, the all-in-one pen is a single device that both measures blood sugar and delivers insulin. Using the all-in-one pen can result in a greater than 50% reduction in time compared to using conventional procedures.

### Example Characterization of a Flexible Glucose Sensor

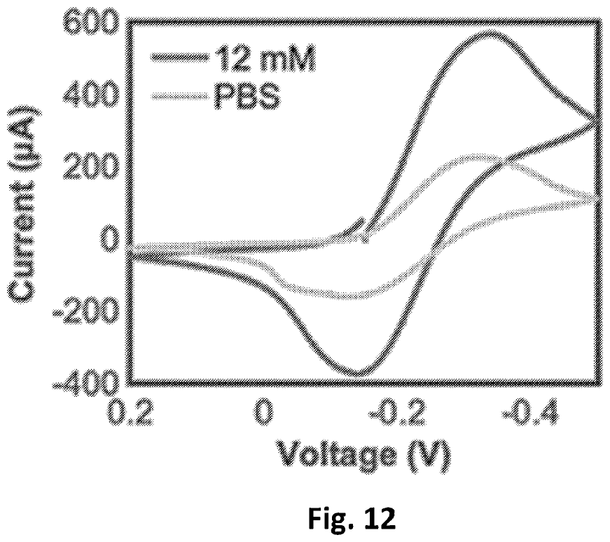

FIG. 12 shows cyclic voltammetry (CV) results of the flexible glucose sensor in a 12 mM glucose solution and in a phosphate buffered saline (PBS). CV performed on the glucose sensor indicated the incorporation of the working electrode's and reference electrode's functionalizations. The CV results showed the sensitivity of the electrodes to glucose with reduction/oxidation peaks that corresponded to the values of about −0.1 V to −0.2 V. An advantage of this modification was that the electrochemical reaction can be monitored at a voltage of 0 V at the working electrode. Operating the working electrode at 0 V reduces the sensor warm-up time and minimizes electrochemical side reactions, as described in more detail below.

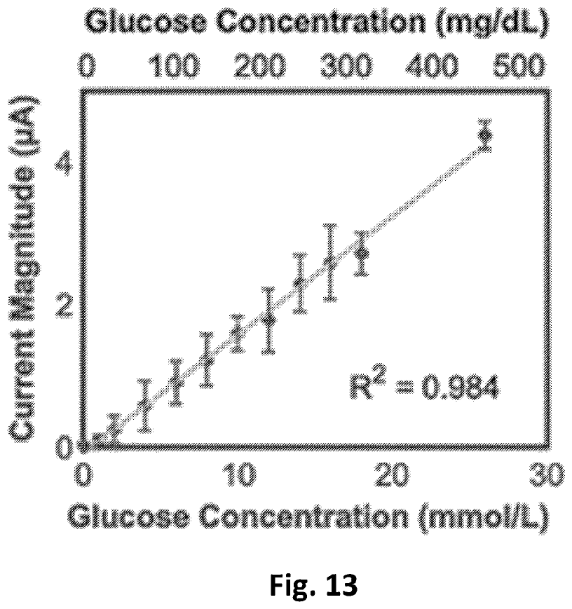

FIG. 13 shows chronoamperometric current measurements versus glucose concentration using a glucose sensor. The performance of the glucose sensor was further characterized using chronoamperometry to measure glucose concentration 15 seconds after the sensor was wetted with the glucose solution. These measurements showed the current response of the sensor remains linear from a glucose concentration of 0 mM to at least 26 mM (468 mg/dL) of glucose (e.g., 0.1 mM, 0.5 mM, 2 mM, 4 mM, 6 mM, 8 mM, 10 mM, 12 mM, 14 mM, 16 mM, 18 mM, 20 mM, 22 mM, 24 mM, or 26 mM) in phosphate-buffered saline (PBS). Therefore, the glucose sensor provided sufficient dynamic range to measure glucose levels expected in patients with diabetes.

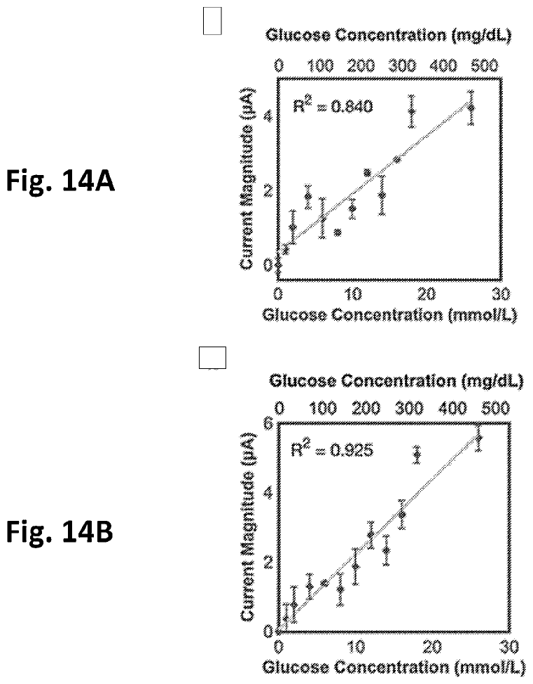

FIGS. 14A and 14B show chronoamperometric current measurements versus glucose concentration using the flexible glucose sensor 5 seconds and 10 seconds after sensor wetting, respectively. Along with the results in FIG. 13, these results show that the sensor's current response to glucose concentration remains linear in the range of 0 mM to at least 26 mM of glucose when using a variety of measurement times between 5 seconds and 15 seconds (e.g., 5 seconds, 10 seconds, or 15 seconds).

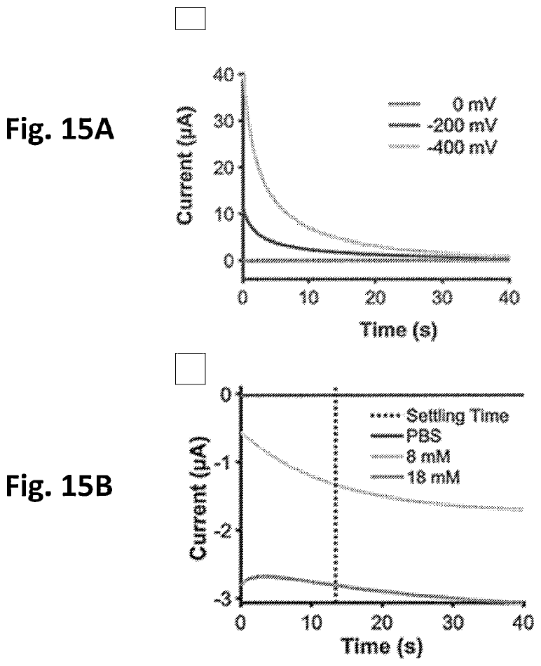

FIG. 15A shows transient currents measured with an all-in-one needle operating at different voltages. These transient current measurements indicate the all-in-one needle's preparation time once the needle's sensor is exposed to a fluid. During this preparation time the all-in-one needle's sensor is wetted and its temperature is stabilized. The preparation time varies with the voltage applied to the working electrode. Applied voltages were 0 V, −0.2 V, or −0.4 V and transient currents were measured by the sensor for 40 seconds. The results indicated that operating the sensor at 0 V substantially reduced preparation time as compared to the other voltages. FIG. 15B shows stabilization time of an all-in-one needle measuring different glucose concentrations. Stabilization time (defined here as when current reaches 90% of its final value) was 13.8 seconds for the glucose sensor, regardless of the glucose concentration measured. The glucose concentrations measured were 8 mM and 18 mM glucose in phosphate-buffered saline (PBS) and were compared to a control sample of PBS without glucose.

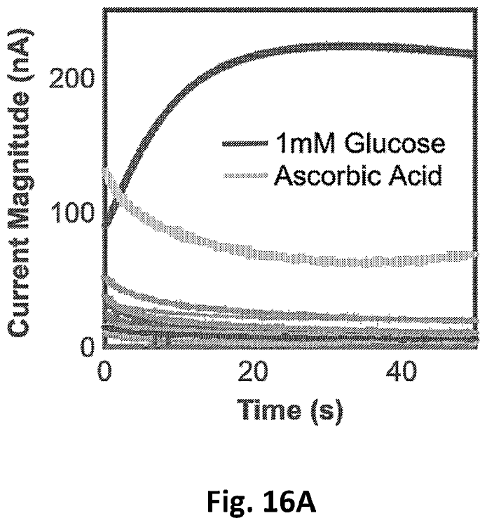

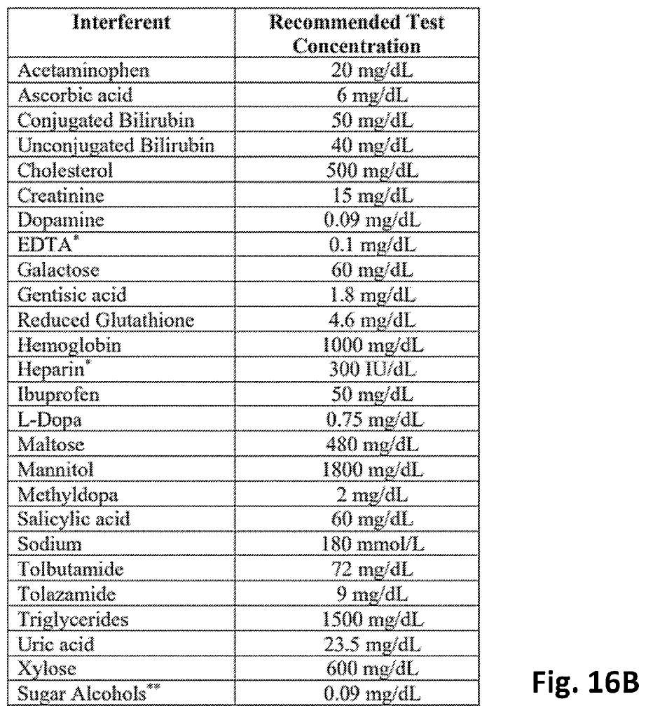

FIG. 16A shows cross sensitivity tests of the flexible glucose sensor. Chronometric measurements at V=0 were conducted to determine the cross-sensitivity of the glucose sensor to the interferents named in the FDA-2013-D-1446-0017 guidelines at the suggested concentrations. One representative trace for each potential interferent was plotted in FIG. 16A. The interferents and their concentrations are in FIG. 16B. The only interferent that had a substantial response was ascorbic acid at a concentration 6-times that of physiological levels, The 6× concentrated ascorbic acid showed a current value that had an amplitude that was about 30% that measured for 1 mM glucose (18 mg/dL). At higher blood glucose concentrations, this interference would be even less significant. Overall, operating the working electrode at 0 V reduces electrochemical side reactions, as evidenced by the minimal cross-sensitivity of the sensor to the approximately 30 interfering substances listed in the FDA guidelines as commonly reported biologically interfering species.

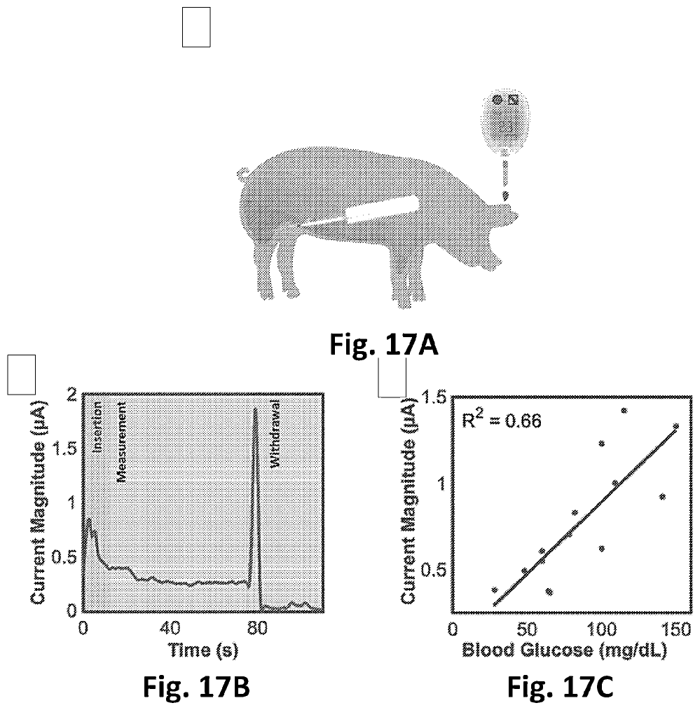

FIG. 17A is a schematic representation of the experimental setup for in vivo testing of the all-in-one needle in a porcine model. The all-in-one needle was tested using chronoamperometric measurements of interstitial glucose levels. The all-in-one needle was compared with a conventional glucometer with a test strip in a non-diabetic animal. Female Yorkshire swine in the range of 60-80 kg were used for testing the all-in-one needle system. The pigs were kept on liquid diet for 24 hours before the procedure and fasted overnight. The pigs were sedated with intramuscular injections of Telazol (2-6 mg/kg) and Xylazine (2-4 mg/kg) and were kept on isoflurane (1-2%) and oxygen (2-3%) either via a face mask or endotracheal tube. For blood sampling, an indwelling catheter was placed in the femoral vein under aseptic conditions. Blood glucose levels by the all-in-one needle were compared to those measured by a commercial TRUEtrack Blood Glucose Meter with testing strips. Measurements using the all-in-one needle were conducted by inserting the all-in-one needle into the thigh of the animal following use of a 16-guage introducer needle. Using the introducer needle is an optional step for measurements in humans but is used for measurements in pigs because of their thicker skin. To prevent hypoglycemia during anesthesia, blood sugar levels were maintained by delivering a bolus of 10 mL of 50% dextrose solution when measured blood glucose was observed to fall below 20 mg/dL.

FIG. 17B shows a chronoamperometric measurement of an all-in-one needle used in vivo. FIG. 17B shows current magnitude vs. time measured as the all-in-one needle was inserted into the subcutaneous tissue, was used to measure glucose concentrations in the interstitial fluid for a period of time, and then was withdrawn from the tissue. Following needle insertion at t=0, the current magnitude first increased rapidly due to wetting and diffusion of the interstitial fluid into the working electrode and then stabilized within 5-15 seconds, yielding a stable measurement current which can be calibrated to a known blood glucose level. When the needle was withdrawn from the tissue, there was a sharp spike corresponding to mechanical perturbation, followed by the current amplitude dropping rapidly to a baseline current similar to that measured prior to insertion.

FIG. 17C shows mean current magnitude 5-10 seconds post insertion versus blood glucose concentration measured with a commercial glucose monitor using a test strip in 14 separate animal measurements. A total of 14 measurements were conducted using separate all-in-one needles on different animals with varied blood glucose levels to generate the in vivo calibration curve shown in FIG. 17C. FIG. 17C shows mean current magnitude in the time windows of 5-10 seconds post insertion vs measured blood glucose. Similar measurements were taken for time windows of 0-5 seconds and 10-15 seconds post insertion. A linear correlation was observed in all 3 time windows, with the 5-10 second time window showing a higher coefficient of determination. This time window may yield the preferable linear response as a balance between giving sufficient time for wetting of the sensor and diffusion of glucose while minimizing depletion of the local concentration of glucose from prolonged electrochemical reactions.

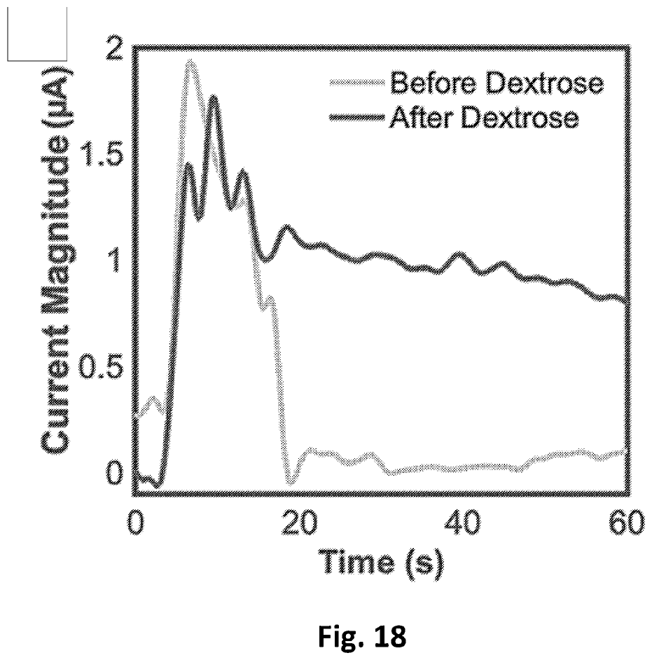

FIG. 18 shows current measurements versus time recorded using the all-in-one needle prior to (blood glucose (BG)=42 mg/dL) and following dextrose injection (BG=105 mg/dL in a non-diabetic animal. Measurements were conducted using two separate all-in-one needle sensors, prior to and 8 minutes following the delivery of a 50% dextrose solution via a femoral catheter. The delay time between measurements was selected to account for the time delay due to diffusion of glucose from blood into the interstitial fluid. Separate recordings in multiple experiments showed an increase in measured current magnitude following dextrose delivery. Blood glucose measurements were recorded simultaneous using a commercial glucometer and corroborated the measurements from the all-in-one needle. These results are consistent with a spike in the pig's blood glucose level induced by the dextrose injection that resulted in a subsequent increase in interstitial glucose concentration. The downward drift during measurement following dextrose delivery is the animal's self-regulation of glucose levels to return to the euglycemic range.

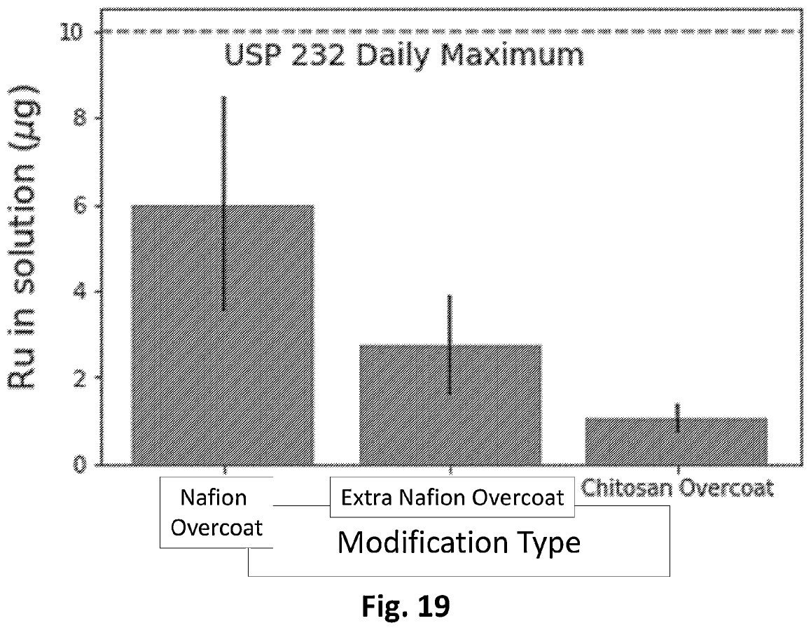

FIG. 19 shows ruthenium (Ru) dissolution from the flexible glucose sensor. To alleviate concerns regarding mediator toxicity, the ruthenium content released by one sensor during transient use (generously estimated to be max of 2 mins) was measured. Inductively coupled plasma mass spectrometry (ICP-MS) was used to measure Ru concentrations. The sensors were prepared with semi-permeable membranes by drop-casting onto the glucose-responsive hydrogel either a 1.5 μL layer of Nafion (0.5% in water) (“Nafion Overcoat”), 3 μL layer of Nafion (0.5% in water) (“Extra Nafion Overcoat”), or 3 μL layer of chitosan (1% in water) (“Chitosan Overcoat”). The modified sensors were then dipped into 2 mL of PBS solution for 2 minutes. The solution was then collected and suspended in a 2% nitric acid matrix for analysis.

The results in FIG. 19 showed that Ru release from the sensor using all three modifications was lower than the daily United States pharmacopeia guidelines (USP 232 Daily Maximum) of 10 μg. Adding additional layers of Nafion or chitosan to the semi-permeable membrane further reduced Ru release by encapsulating Ru. The Ru release from the sensor is low enough that the all-in-one needle may be used at least four times per day without exposure to Ru concentrations higher than the guidelines.

### Conclusion

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one clement of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A glucose sensor configured to be wrapped around a surface of an injection needle or cannula, the glucose sensor comprising:
a flexible substrate;
at least two electrodes disposed on a surface of the flexible substrate;
a glucose-responsive hydrogel at least partially disposed on a first electrode of the at least two electrodes; and
a membrane permeable to glucose;
wherein:
the membrane is disposed on the glucose-responsive hydrogel; and
a total thickness of the glucose sensor is less than 10 μm.

2. The glucose sensor of claim 1, wherein the glucose sensor is configured to measure a glucose concentration up to about 26 mM.

3. The glucose sensor of claim 2, wherein the glucose sensor is capable of measuring a glucose concentration of a patient when the injection needle or cannula is inserted into the patient in less than 40 seconds after the injection needle or cannula is inserted.

4. The glucose sensor of claim 1, wherein the glucose-responsive hydrogel comprises:
a polymer permeable to glucose;
an oxidoreductase enzyme that catalyzes oxidation of glucose; and
a redox mediator.

5. The glucose sensor of claim 4, wherein the polymer comprises at least one of chitosan, polyvinyl alcohol, or Nafion.

6. The glucose sensor of claim 4, wherein the oxidoreductase enzyme comprises at least one of glucose oxidase or a derivative or mutant thereof.

7. The glucose sensor of claim 4, wherein the redox mediator comprises at least one of an iron salt complex, an osmium salt complex, or a ruthenium salt complex.

8. The glucose sensor of claim 7, wherein the iron salt complex comprises potassium ferricyanide.

9. The glucose sensor of claim 7, wherein the ruthenium salt complex comprises hexamine ruthenium.

10. The glucose sensor of claim 1, wherein the at least two electrodes comprise a noble metal.

11. The glucose sensor of claim 1, wherein the membrane comprises Nafion.

12. The glucose sensor of claim 1, wherein the flexible substrate comprises at least one of polydimethylsiloxane or polyimide.

13. The glucose sensor of claim 1, wherein the flexible substrate is a first flexible substrate and further comprising:
a second flexible substrate disposed on at least a portion of the first flexible substrate, the second flexible substrate having voids where the at least two electrodes are located.

14. The glucose sensor of claim 1 further comprising the injection needle, wherein the flexible substrate is disposed on a shaft of the injection needle.

15. An all-in-one insulin pen comprising the glucose sensor of claim 14.

16. A method of measuring a glucose concentration of a patient comprising:
inserting an injection needle into the patient, the injection needle fluidically coupled to a container holding a medicine; and
measuring a glucose concentration of the patient from an interstitial fluid of the patient in less than 40 seconds after the injection needle is inserted into the patient using a glucose sensor wrapped around an outer surface of the injection needle.

17. The method of claim 16, further comprising:
administering the medicine to the patient through the injection needle if the glucose concentration measured by the glucose sensor during the step of measuring is outside of a euglycemic range;
wherein the step of administering is performed without withdrawing the needle after the step of measuring.

18. The method of claim 16, wherein the step of measuring comprises operating the glucose sensor at a voltage of about −0.4 V to about 0.4 V.

19. The method of claim 16, wherein the step of measuring comprises measuring a glucose concentration of up to about 26 mM.

20. A glucose sensor wrapped around an outer surface of an injection needle, the glucose sensor comprising:
a flexible polymer substrate;
a membrane permeable to glucose; and
a patterned metal electrode disposed on the flexible polymer substrate and coated with a glucose-responsive hydrogel, the glucose-responsive hydrogel comprising:
glucose oxidase;
chitosan; and
hexamine ruthenium;
wherein:
the membrane is disposed on the flexible polymer substrate;
the glucose sensor is capable of measuring a one-time glucose concentration of a patient when the injection needle is inserted into the patient; and
the glucose sensor is capable of measuring the one-time glucose concentration in less than 40 seconds after the injection needle is inserted into a patient.
